(19) United States
(12) Patent Application Publication
(10) Pub. No.: US 2004/0103001 A1
(43) Pub. Date: May 27, 2004

(54) System and method for automatic diagnosis of patient health

(76) Inventors: Scott Thomas Mazar, Inver Grove Heights, MN (US); Yatheendhar Manicka, Woodbury, MN (US); Bruce H. Kenknight, Maple Grove, MN (US); Richard Fogoros, Pittsburgh, PA (US); Michael J. Pederson, Minneapolis, MN (US)

Correspondence Address: MERCHANT & GOULD PC, P.O. BOX 2903, MINNEAPOLIS, MN, 55402-0903, US

(21) Appl. No.: 10/306,855

(22) Filed: Nov. 26, 2002

(51) Int. Cl. G06F 17/60; A61B 5/00

(52) U.S. Cl. 705/2; 600/300

(57) ABSTRACT

Methods and systems for providing a clinically modeled automatic diagnosis of patient health are disclosed. A preferred embodiment uses a medical device and network to analyze patient data in a manner consistent with a standard of medical care. Some embodiments of a system disclosed herein also can be configured as an Advanced Patient Management System that helps better monitor, predict and manage chronic diseases.

### TECHNICAL FIELD

The present system relates generally to an Advanced Patient Management System and particularly, but not by way of limitation, to such a system that can automatically diagnose patient health by analyzing sensed patient health data in comparison to population data to yield a multi-dimensional health state indication and disease trend prediction.

### BACKGROUND

According to Plato, “Attention to health is life's greatest hindrance.” Historians believe Plato was bemoaning the physical limitations of his body that prevented complete devotion to thought. However, attention to health in the modern world is also limited constrained by the physical burdens placed on clinicians to digest and synthesize increasing amounts of medical data, and by the fiscal burdens of a modern healthcare system desperate to contain costs through the use of HMOs and other capitated cost devices.

Over the past 20 years, medical care costs have risen annually at over twice the rate of inflation compared to the rest of the economy. A major cost factor is the time and expense incurred in evaluating patient health in traditional health care settings—i.e., the physician's office or a hospital. To stem the tide of rising costs, physicians and other health care professionals must strike a reasonable balance between containing costs and providing quality medical care—an often difficult balance when facing the challenges of treating chronic disease.

Modern medicine generally categorizes diseases as either chronic or acute. Chronic diseases such as chronic heart disease, hypertension and diabetes often require a regular treatment schedule for the duration of the patient's life. Chronic diseases also have the tendency to spawn other health care problems. For example, chronic heart disease often causes edema and other circulatory problems that require treatment modalities distinct from the treatment of the chronic heart problem. Diabetes often leads to neuropathy and eventual amputation. Thus, physicians treating chronic illnesses devote most of their time and resources to managing rather than curing the disease.

In contrast to chronic diseases, acute diseases are typically manifested by a sudden or severe appearance of symptoms or a rapid change or worsening of patient condition. Acute diseases often require immediate and often costly medical intervention. However, acute episodes may be suitable for management to the extent they are predictable or relate to a chronic condition.

Disease management may be defined as managing a patient with a known diagnosis with the intention of providing patient education and monitoring to prevent or minimize acute episodes of the disease. Reducing or eliminating the number of acute episodes in turn reduces or eliminates medical costs and also improves a patient's sense of subjective well-being. Treating physicians have observed that subjective feelings of well-being often correlate with objective improvements in patient health and serve as a useful predictive health management and assessment tool. In sum, disease management places greater emphasis on preventive, comprehensive care to monitor disease trends that might help improve the health of entire populations of patients.

With advances in genetic testing (analyzing an individual's genetic material to determine predisposition to a particular health condition or to confirm a diagnosis of genetic disease), disease management can take the form of coordinated patient care from birth to death. In this cradle to grave approach, physicians not only manage patients with clinically manifest diseases or symptoms, but also patients that seem perfectly healthy.

However, to effectively manage a chronic, acute or predisposed disease state, a physician must first make a proper diagnosis. Diagnosis is defined as the art or act of identifying a disease from its signs and symptoms. A physician seeking data about a patient to form a diagnosis will invariably subject the patient to one or more diagnostic procedures, e.g., blood or urine assays. Typically, a medical technician draws blood or procures urine from the patient. The sample is then analyzed in a manner that generates considerable amounts of data about the sample. However, an accurate diagnosis often requires the gathering and analysis of patient health data from sources other than sample data, including the patient's medical history and prevailing trends in medical practice and treatment. As a result, the physician is challenged to synthesize the collected information into a cohesive and meaningful diagnosis. Since the quality of this synthesis depends in large part on the skill and education of the physician, the potential for error, or misdiagnosis, can be significant. If the data fails to reveal a symptom or disorder within the scope of the physician's knowledge, then the physician could misdiagnose the problem. In addition, physician bias can result in the misuse or misunderstanding of sample data.

One way to minimize physician error and/or misdiagnosis is to automate at least part of the diagnostic process. Automation is possible because much of the practice of modern medicine can be reduced to algorithmic expressions. That is, the diagnosis of a health problem often follows a sequence of steps that serve to isolate the cause of the problem. Advanced cardiac life support (ACLS) and advanced trauma life support (ATLS) methodologies have shown how much patient care can be improved by setting standards of care. Some standards may be translated into clinical algorithms, which provide an objective, computer-accessible framework for the standard of care. In the past, the treating physician was the key repository of a patient's medical information and often the only person capable of giving it clinical meaning. Now computer technologies can partially automate this process.

Physicians and other health care professionals now recognize that almost all “knowledge based” clinical reasoning can be performed better and more reliably by computers. However, the quality of that clinical reasoning depends on the quality of the artificial intelligence parameters programmed into the computer. At its most basic level, artificial intelligence can be defined as the manipulation of raw data input. However, when raw data is given structure or order, that data is transformed into information. In other words, the raw data has been distilled into something meaningful. The process of compiling meaningful information is the first step in creating a base of knowledge. As computer-based systems become more knowledgeable, such systems can, by using algorithms that reflect real-world parameters, develop the ability to make discriminating judgments on subsequent data input. By organizing data in a way that allows a computer-based system to develop judgment, the system has made the first step in obtaining what might be called wisdom.

In the field of computer analysis of medical or patient health data, principles of fuzzy logic can be employed to approximate human wisdom. In basic terms, fuzzy logic addresses the likelihood of certain probabilities instead of absolute values that are characteristic of Boolean logic. Optimally, fuzzy logic “is so determinative in its constituent distinctions and relations as to convert the elements of the original situation into a unified whole.” John Dewey, Logic: The Theory of Inquiry, 1938. By unifying the disparate elements of clinical diagnosis with fuzzy logic principles, the resulting output more closely approaches a clinically acceptable standard of medical care reflecting the wisdom gained by clinical practice and experience.

In addition, a system that automates diagnostic processes should have a significant competitive advantage in a capitated health care environment. Such a system should be able to analyze patient data to automatically identify very critical points in any disease process so that intervention is economically, clinically and humanistically maximized.

Thus, disease management using automated diagnosis is a revolutionary step in the practice of medicine. Because of the rapid advances in miniaturized computer technology, pioneering advances in disease management are now possible. In the past, the treating physician was not only the key repository of a patient's medical information, but large segments of that information were lost when the physician died. Now, diagnostic and data storage functions can be partially automated and preserved by using computer technologies, which provides the means to present and preserve medical knowledge in an orderly, temporal fashion.

However, such automatic diagnoses may be limited by a patient's or clinician's access to systems capable of quickly and efficiently providing the diagnosis. Automatic diagnosis is of little value in terms of reducing costs and improving efficiency if the clinician's use of computer technology is limited to traditional settings like the doctor's office or the hospital. In addition, relying on patient visits as the primary means of collecting patient information is often unreliable as many patients fail to make or keep regularly scheduled appointments.

Thus, for these and other reasons, there is a need for an Advanced Patient Management System comprising or configured as a Data Management System capable of storing and efficiently analyzing patient health data to provide a clinically modeled automatic diagnosis of patient health that is determinative in its constituent distinctions and relations and easily accessible by the patient or the physician. In this way, the Data Management System will lower the cost of medical care and reduce the analytical burdens on clinicians faced with increasing amounts of clinical data.

### SUMMARY

According to one aspect of the invention, there is provided a system and method for automatic diagnosis of patient health using a Data Management System that might comprise a component of, or be configured as, a more comprehensive Advanced Patient Management (APM) system. The Data Management System comprises a medical device component and a network component. The medical device component includes an implantable medical device, and the network component includes either a linear or non-linear analysis network. A non-limiting example of such a non-linear analysis network is a fuzzy logic system. As used herein, “multi-dimensional indication of patient health,” “initial evaluation of patient health,” “patient health evaluation,” “analyzed patient health data,” “preliminary evaluation,” and “automatic diagnosis” are substantively synonymous terms of varying scope. For example, a “multi-dimensional indication of patient health” is conceptually broader than a “preliminary evaluation”—the latter obtained through further algorithmic analysis of multi-dimensional data. Nevertheless, all the aforementioned terms represent a systematic evaluation of patient health based on a clinically derived algorithmic analysis of patient data that reflects a standard of medical care. Also, as used herein, a “clinician” can be a physician, physician assistant (PA), nurse, medical technologist, or any other patient health care provider.

In one embodiment, the device component of the Data Management System comprises a data evaluation system and the network component comprises a data teaching system. The data evaluation system further comprises: a sensing module; a data management module; an analysis module and a communications module.

The sensing module is adapted to sense patient health data. Patient health data may comprise any physiological parameter suitable for measurement by the sensing module. By way of non-limiting example only, such physiological parameters include the patient's body temperature, the time it takes for a human heart to complete a cardiac cycle (similar to the way a pacemaker functions) or patient activity.

The data management module is adapted to store and archive patient data, sensed patient health data and patient population data. Patient data can comprise any statistic, measurement or value of patient health coded for algorithmic medical diagnosis or analysis. Such coded patient data can be downloaded to the data management module to populate a database of historical patient information. Also, patient data in the form of patient health data sensed from the patient can be downloaded to the data management module to populate a database or the historical patient database. Patient data in the form of patient population data can comprise data from similarly sick patients or genetically similar patients. Such patient population data also can be downloaded to the data management module to populate the module with a patient population database.

The analysis module is adapted to score or analyze the patient population data relative to the sensed and/or historical patient data using clinically derived algorithms to yield a multi-dimensional indication of patient health. Such analysis may take the form of correlating patient health data using known data correlation techniques. The clinically derived algorithms can be customized to reflect a standard of medical care. By way of non-limiting example only, the analysis module can include algorithms reflecting clinical methodologies used at the Mayo Clinic to assess and treat cardiac arrhythmias. By way of further non-limiting example only, the analysis module can include algorithms reflecting clinical methodologies used at the Cleveland Clinic to assess and treat hormonal disorders. Other clinical methodologies that have been or can be reduced to algorithmic expression may be used or combined with other clinical methodologies to analyze patient health data. In this way, the system can be fine-tuned to reflect a local or regional standard of medical care or a standard of care specifically customized to the patient's needs. Moreover, by using clinically derived algorithms that express a standard of medical care, there is consistent delivery of quality of health care. Such consistency serves to improve the cost-effectiveness of medical care by offloading the diagnostic burden placed on the clinician to the Data Management System. The multi-dimensional indication of patient health may comprise a prediction of a disease trend, a prediction of a next phase of disease progression, a prediction of co-morbidities, an inference of other possible disease states, a prediction of a trend of patient health or other clinical trajectories.

The communications module is adapted to communicate the scored or analyzed data and patient health evaluation to a physician or other clinician for further evaluation and analysis. The communications module also is adapted to communicate the scored or analyzed data and patient health evaluation to the data management module or a fuzzy logic analysis network for future diagnoses or teaching purposes. The communications module is further adapted to communicate the scored or analyzed data and patient health evaluation to a patient.

The data teaching system of the Data Management System comprises an analysis network, including a neural network (or equivalent) system. The neural network comprises a centralized repository of relevant clinical data accessible by the data evaluation system. The neural network comprises patient data databases reflecting historical symptoms, diagnoses and outcomes, along with time development of diseases and co-morbidities. The neural network analyzes the data to find clinically useful correlations between data sets and create a series of outputs. Moreover, as new clinical information is sensed, analyzed and communicated by the data evaluation system, that information is communicated to the neural network. Thus, the neural network can be adapted to constantly upgrade its knowledge databases with new clinical information to improve the diagnostic accuracy of the system by increasing its ability to make accurate discriminating judgments.

In another embodiment, patient data is analyzed under principles of fuzzy logic in contrast to more deterministic Boolean models. Fuzzy logic is known to handle the concept of partial truth—truth values between “completely true” and “completely false.” The process of “fuzzification” is a methodology to generalize any specific theory from a crisp (discrete) to a continuous (fuzzy) form.

In a preferred embodiment of the system and method for the automatic diagnosis of patient health using a medical device and network configured as a Data Management System capable of applying principles of fuzzy logic to clinically derived algorithms to analyze patient data in a manner consistent with a standard of medical care, the medical device is internal to the patient and may comprise, in whole or in part, the data evaluation system comprising the sensing, data management, analysis and communications modules and the data teaching system. The neural network, in a preferred embodiment of the system, comprises computer accessible patient data, historical data and patient population data of similarly sick and genetically similar patients.

The various embodiments described above are provided by way of illustration only and should not be construed to limit the invention. Those skilled in the art will readily recognize various modifications and changes that may be made to the present invention without following the example embodiments and applications illustrated and described herein, and without departing from the true spirit and scope of the present invention, which is set forth in the following claims.

### BRIEF DESCRIPTION OF THE DRAWINGS

In the drawings, which are not necessarily drawn to scale, like numerals describe substantially similar components throughout the several views. Like numerals having different letter suffixes represent different instances of substantially similar components. The drawings illustrate generally, by way of example, but not by way of limitation, various embodiments discussed in the present document.

FIG. 1 is a schematic/block diagram illustrating generally, among other things, one embodiment of the system and method for automatic diagnosis of patient health of the present invention.

FIG. 2 is a schematic/block diagram illustrating generally, among other things, another embodiment of the system and method for automatic diagnosis of patient health of the present invention.

FIG. 3 is a schematic/block diagram illustrating generally, among other things, another embodiment of the system and method for automatic diagnosis of patient health of the present invention.

FIG. 4 is a schematic/block diagram illustrating generally, among other things, another embodiment of the system and method for automatic diagnosis of patient health of the present invention.

FIG. 5 is a schematic/block diagram illustrating generally, among other things, another embodiment of the system and method for automatic diagnosis of patient health of the present invention.

FIG. 6 is a schematic/block diagram illustrating generally, among other things, another embodiment of the system and method for automatic diagnosis of patient health of the present invention.

### DETAILED DESCRIPTION

In the following detailed description, reference is made to the accompanying drawings that form a part hereof, and in which are shown by way of illustration specific embodiments or examples. These embodiments may be combined, other embodiments may be utilized, and structural, logical, and electrical changes may be made without departing from the spirit and scope of the present invention. The following detailed description is, therefore, not to be taken in a limiting sense, and the scope of the present invention is defined by the appended claims and their equivalents.

The present system and method are described with respect to a medical device and network configured as a Data Management System capable of automatically diagnosing patient health using clinically derived algorithms that reflect a standard of medical care. The diagnosis made by the present system is best understood as an initial evaluation of patient health that provides a starting point for further evaluation, analysis or confirmation by a physician or other health care professional. Moreover, because the system is adapted to automatically sense patient health data on a regular basis, the system provides a sample of clinically relevant information that greatly exceeds the amount of information the physician might obtain during office visits by the patient, which are often infrequent and irregular. In providing an initial evaluation of patient health, the system reduces the amount of data collection and review by the clinician. This helps reduce costs and improve the management of the patient and the patient's disease.

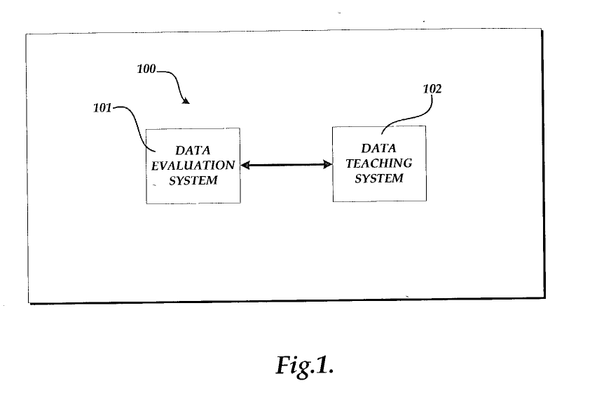

FIG. 1 is a schematic/block diagram illustrating generally an embodiment of the Data Management System 100 capable of automatically diagnosing patient health using clinically derived algorithms. The system further comprises data evaluation 101 and data teaching 102 systems.

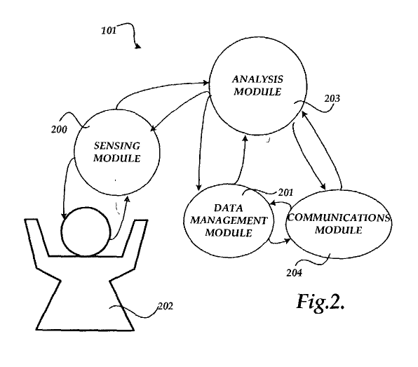

FIG. 2 is a schematic/block diagram illustrating generally an embodiment of the data evaluation system 101 of the Data Management System 100 comprising: a sensing module 200 adapted to sense data from a patient 202; a data management module 201 adapted to store and archive data; an analysis module 203 adapted to analyze data to make an initial evaluation of patient health; and a communications module 204 adapted to communicate the analyzed data and initial evaluation of patient health.

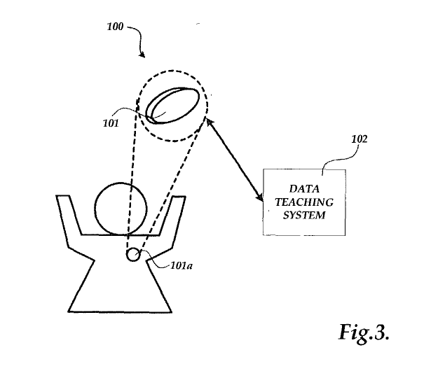

In one embodiment, as illustrated in FIG. 3, the data evaluation component 101 of the Data Management System 100 comprises an implantable medical device 110a. In this embodiment, the implantable medical device comprises the sensing 200, data management 201, analysis 203 and communications 204 modules illustrated in FIG. 2.

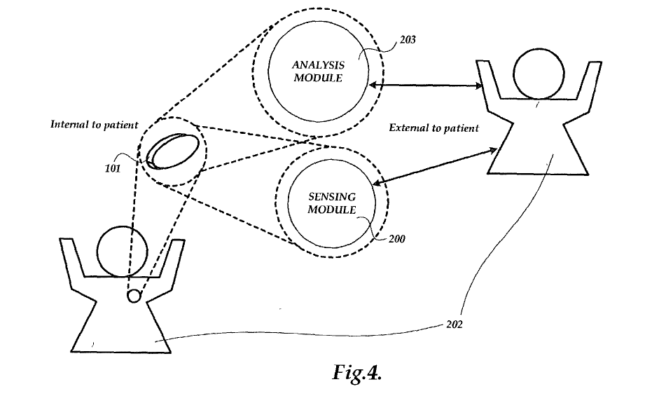

FIG. 4 is a schematic/block diagram illustrating generally an embodiment of the data evaluation component 101 of the Data Management System 100, wherein the sensing 200 and analysis 203 modules of the data evaluation component 101 comprise a combination of internal and external modules. For example, the sensing module 200 can be internal to the patient 202 while the analysis module 203 is external to the patient. In another example, the sensing module 200 can be external to the patient 202 while the analysis module 203 is internal. In addition, both sensing 200 and analysis 203 modules can be either internal or external. Those skilled in the art will appreciate that various internal and external configurations of the sensing 200 and analysis 203 modules are possible without departing from the spirit and scope of the invention.

The sensing module 200 is adapted to sense patient health data. Patient health data can comprise internal or external patient data, i.e., cardiovascular data, electrochemical data, blood chemistry data, temperature, wedge pressure, oxygen saturation, weight, subjective well-being input, blood pressure, EKG data or any other physiological parameter suitable for measurement by the sensing module 200.

The data management module 201 is adapted to store and archive patient data for contemporaneous and future analysis. Patient data might comprise patient health data, historical patient data and patient population data. Historical patient data can comprise cumulative patient health data sensed or collected from the patient on a regular basis over a period of time or coded patient health data. Patient population data might comprise data from populations of similarly sick or genetically similar patients or both. The data management module 201 is also adapted for data retrieval by the communications module 204.

The communications module 204 is adapted to retrieve data from the data management module 201 on a periodic basis for analysis by the analysis module 203. The communications module 204 also is adapted to communicate the sensed or analyzed patient data to the data management module 201 and/or the neural network 500. This allows the data management module 201 to utilize the sensed or analyzed patient data in subsequent evaluations of patient health and allows the neural network 500 to automatically update its databases with the most recent patient data. The communications module 204 is further adapted to communicate the sensed or analyzed data to a physician 501 or other healthcare clinician. In this way, the communications module 204 can communicate to the physician 501, a clinician or the patient 202 a relative urgency of intervention based on the preliminary evaluation.

The analysis module 203 is adapted to receive patient data from the communications module 204 and score or analyze that data in reference to patient population data using clinically derived algorithms that reflect or embody a standard of medical care. Such standards of medical care can reflect the institutional practices and methodologies of institutions like, by way of non-limiting example only, the Cleveland Clinic, the Mayo Clinic or the Kaiser Permanente system, that have been reduced to algorithmic expression.

The comparative analysis of patient health in view of a standard or standards of practicing medicine yields a multi-dimensional evaluation of patient health or preliminary evaluation firmly rooted in clinical practice. Such comparative analysis may be accomplished by the correlation of patient health data using known data correlation techniques like, by way of non-limiting example only, multiple regression analysis, cluster analysis, factor analysis, discriminate function analysis, multidimensional scaling, log-linear analysis, canonical correlation, stepwise linear and nonlinear regression, correspondence analysis, time series analysis, classification trees and other methods known in the art. The multi-dimensional evaluation includes a prediction of a disease trend, a prediction of a next phase of disease progression, a prediction of co-morbidities, an inference of other possible disease states, a prediction of a trend of patient health or other clinical trajectories.

To make a preliminary evaluation, the Data Management System 100 uses clinically derived algorithms to match patient data to clinical outcomes. The algorithms can be the result of the extraction, codification and use of collected expert knowledge for the analysis or diagnosis of medical conditions. For example, the algorithms can comprise institutional diagnostic techniques used in specific clinical settings. By reducing the diagnostic methodologies of institutions like the Cleveland Clinic, the Mayo Clinic or the Kaiser Permanente system to algorithmic expressions, a patient will have the benefit of the diagnostic expertise of a leading medical institution without having to visit the institution. Since the standard of medical care is often viewed as a local or regional standard, the Data Management System 100 can allow the physician to select the diagnostic techniques or methodologies of a specific institution or combination of institutions that best reflect the local or regional standard of care or the specific needs of the patient.

In practice, an algorithmic analysis of contemporaneous patient health data in comparison to historical patient data might yield an initial or preliminary evaluation of patient health that predicts patient health degradation and disease progression. This initial diagnosis is then communicated to the physician 501 for further evaluation, analysis or confirmation.

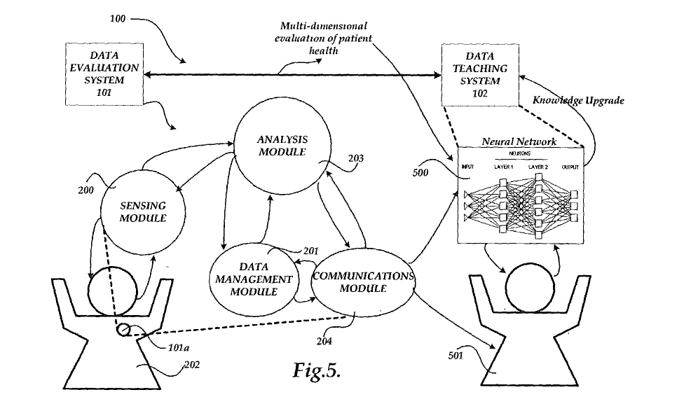

FIG. 5 is a schematic/block diagram illustrating generally an embodiment of the Data Management System 100. In this embodiment, the data evaluation component 101 of the Data Management System 100 is primarily an implantable medical device 101a comprising, in whole or in part, the sensing 200, data management 201, communications 204 and analysis modules 203. After implantation of the medical device 101a, the sensing module 200 is adapted to sense physiological data. That data, for example, cardiovascular function, is electronically transmitted to the data management module 201 via the communications module 204. The data management module 201 is adapted to store the sensed physiological (patient) data. The data management module 201 can include patient population data of similarly sick or genetically similar patients in addition to historical and coded patient data. Contemporaneous physiological data is then analyzed and compared against historical patient data and/or patient population data using clinically derived algorithms of patient health that reflect or embody a standard of medical care. In this way, an initial evaluation of patient health is made by using the clinically derived algorithms to assess the patient's current health status in comparison to objective or historical patient data. As further illustrated in FIG. 5, this initial evaluation of patient health is then communicated to the physician 501 for further evaluation, analysis or confirmation via the communications module 204. In this embodiment, communication might be accomplished by transmitting patient data to a neural network 500 accessible by the physician 501. The physician 501 may further evaluate the preliminary evaluation for urgency of intervention or other factors. In addition, the physician's evaluation can be communicated to the neural network 500 to populate its databases with contemporaneous patient data to improve the accuracy of future initial evaluations of patient health.

As illustrated in FIG. 5, the data teaching system 102 comprises a neural network 500 (or equivalent) system. In the abstract, neural networks are analytic techniques modeled after hypothesized processes of learning in the cognitive system and the neurological functions of the brain. Neural networks are capable of predicting new observations (on specific variables) from other observations (on the same or other variables) after executing a process of so-called learning from existing data. Neural networks are often described as comprising a series of layers further comprising a set of neurons. One of the major advantages of neural networks is their ability to approximate any continuous function.

In one embodiment, the neural network 500 comprises a collection of historical symptoms, diagnoses and outcomes, along with time development of the diseases and co-morbidities. This collection of clinical data may be coded and input into the neural network 500 to populate the network 500 with an initial clinical database from which may be derived a set of baseline health evaluation outputs. In this way, the neural network 500 of the present intervention can be partially trained with clinical information. Alternatively, the neural network's 500 clinical database may comprise contemporaneously sensed and stored patient health data. In either configuration, the neural network 500 has the ability to capture a time dependent dimension of disease state progression. Thus, when new clinical information is presented to the neural network 500, the network creates new neural network coefficients that can be distributed as a neural network or data teaching system 102 knowledge upgrade. By constantly updating the neural network 500 with patient data, the neural network 500 is adapted to changing clinical parameters. The neural network 500 of the present invention also comprises means to verify neural network conclusions for clinical accuracy and significance. The neural network 500 further comprises a database of test cases, appropriate outcomes and the relative occurrence of misidentification of the proper outcome or diagnosis. The neural network 500 is further adapted to establish a threshold of acceptable misidentifications or misdiagnoses.

In one embodiment, the neural network 500 performs, in whole or in part, the analytical function of the system 100 and is configured to approximate the knowledge of a physician and a standard of medical care by making discriminating judgments based on a probable cause of a disease determined through the analysis of patient health data in view of a set or sets of clinical methodologies. One way to analyze this medical data is to use principles of fuzzy logic. Fuzzy logic, in contrast to more deterministic Boolean models, provides analytical output of medical data sets in terms of clinical probabilities as compared to more rigid absolutes.

Just as there is a strong relationship between Boolean logic and the concept of a subset, there is a similar strong relationship between fuzzy logic and fuzzy subset theory. In classical set theory, a subset U of a set S can be defined as a mapping from the elements of S to the elements of the set {0, 1}, U: S→{0, 1}. This mapping may be represented as a set of ordered pairs, with exactly one ordered pair present for each element of S. The first element of the ordered pair is an element of the set S, and the second element is an element of the set {0, 1}. The value zero is used to represent non-membership, and the value one is used to represent membership. Thus, the truth or falsity of the statement, x is in U, is determined by finding the ordered pair whose first element is x. The statement is true if the second element of the ordered pair is 1, and the statement is false if it is 0.

Similarly, a fuzzy subset F of a set S can be defined as a set of ordered pairs, each with the first element from S, and the second element from the interval [0,1], with exactly one ordered pair present for each element of S. This defines a mapping between elements of the set S and values in the interval [0,1]. The value zero is used to represent complete non-membership, the value one is used to represent complete membership, and values in between are used to represent intermediate degrees of membership. The set S is referred to as the “Universe Of Discourse” for the fuzzy subset F. Frequently, the mapping is described as a function, the membership function of F. Thus, the degree to which the statement, x is in F, is true is determined by finding the ordered pair whose first element is x. The degree of truth of the statement is the second element of the ordered pair. In practice, the terms “membership function” and fuzzy subset are used interchangeably.

Because the data evaluation system 101 includes analytical capabilities that exceed the more rigid, deterministic outcomes characteristic of rule-based systems, the data evaluation system 101, although capable of rigid, deterministic output, is also a capable of assessing clinical probabilities. By way of non-limiting example only, when operating in fuzzy logic or probabilistic mode, the data evaluation system 101 may report an 80% level of confidence in its preliminary evaluation of patient health. In addition, the data evaluation system 101 might also query the clinician 501 or patient 202 for more information to further refine the preliminary evaluation. The data evaluation system 101 also might advise the clinician 501 or patient 202 that it requires more patient test data to accurately assess the affect of sensed patient data on a projected co-morbidity. The data evaluation system 101 might further advise action to be taken on the medical device to modify or refine its sensing capabilities. In either deterministic or probabilistic mode, the analytical output of the data evaluation system 101 can be used to upgrade the neural network knowledge base in a manner that allows the data evaluation system 102 to become smarter, and hence more accurate, as it analyzes and gains greater access to patient data.

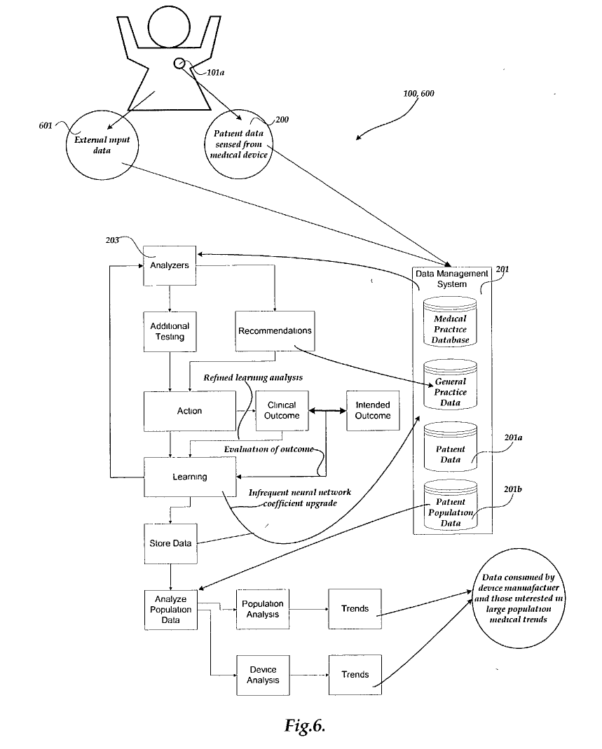

The Data Management System 100 of the present invention can be configured as an Advanced Patient Management system (APM) 600. FIG. 6 is a schematic/block diagram illustrating generally an embodiment of the Data Management System 100 configured as an APM system 600. In this configuration, the analytical function of the system 100, 600 can be viewed as an electronic doctor or eDoC™ with diagnostic capabilities approaching the knowledge and intelligence base of a clinician.

APM is a system that helps patients, their physicians and their families to better monitor, predict and manage chronic diseases. In the embodiment shown in FIG. 6, the APM system 600 consists of three primary components: 1) an implantable medical device (ICD, pacemaker, etc.) 101a with sensors 200 adapted to monitor physiological functions, 2) a Data Management System 201, adapted to process the data collected from the sensors, and 3) eDOc™, an analytical engine 203 adapted to combine device collected data with externally available data 601 from patients' medical records, external devices, etc. APM is designed to support physicians and other clinicians in using a variety of different devices, patient-specific and non-specific data, along with medication therapy, to provide the best possible care to patients.

Currently, implanted devices often provide only therapy to patients. APM moves the device from a reactive mode into a predictive one, so that in addition to providing therapy to the patient, it collects information on other physiological indicators. By way of non-limiting example only, other physiological indicators include blood oxygen levels, autonomic balance, etc. That data is combined with patient-specific externally collected data 601, from, by way of non-limiting example only, a scale, a pulse oxymeter, etc. and trended. By combining internal 200 and external measurements 601 with historical information 201a, 201b, physicians and other clinicians can use APM to develop predictive diagnoses.

When the Data Management System 100 is adapted to operate as an eDOc™, it significantly reduces the amount of data presented to the physician 501 for diagnostic analysis, which saves time and money. The Data Management System 100 also changes raw data into useful information. By using computer technologies in this manner, the clinician is able to synthesize and give clinical meaning to much more data than he or she would normally be capable of handling.

It is to be understood that the above description is intended to be illustrative, and not restrictive. For example, the above-described embodiments may be used in combination with each other. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.”

What is claimed is:

1. A method for using a data management system to automatically diagnose patient health comprising the steps of:
a. populating a data management module adapted to store and archive data with patient population data;
b. sensing data from a patient using a medical device;
c. delivering the patient data from the medical device to the data management module;
d. retrieving data from the data management module for analysis;
e. analyzing the retrieved data using a neural network comprising clinically derived algorithms reflective of a standard of medical care to provide an initial evaluation of probable patient health based on the analyzed data; and
f. communicating the sensed data, the analyzed data and the patient health evaluation.

2. The method of claim 1, wherein the steps are performed electronically.

3. The method of claim 1, wherein the step of populating the data management module with patient population data comprises the further step of populating the data management module with external patient data.

4. The method of claim 1, wherein the step of populating the data management module with patient population data comprises the further step of populating the data management module with historical data from the patient.

5. The method of claim 1, wherein the step of populating the data management module with patient population data comprises the further step of populating the data management module with data comprised of similarly sick patients.

6. The method of claim 1, wherein the step of populating the data management module with patient population data comprises the further step of populating the data management module with data comprised of genetically similar patients.

7. The method of claim 1, wherein the step of sensing data from a patient using a medical device comprises the further step of sensing data using a medical device internal to the patient.

8. The method of claim 1, wherein the step of sensing data from a patient using a medical device comprises the further step of sensing data using a medical device external to the patient.

9. The method of claim 1, wherein the step of retrieving data from the data management module comprises the further step of retrieving data periodically.

10. The method of claim 1, wherein the step of analyzing the retrieved data comprises the further step of scoring the patient data in reference to patient population data.

11. The method of claim 1, wherein the step of analyzing the retrieved data comprises the further step of scoring the patient data in reference to patient population data using principles of fuzzy logic.

12. The method of claim 10, wherein the step of scoring the patient data in reference to patient population data comprises the further step of scoring the data to yield a multi-dimensional indication of patient health.

13. The method of claim 11, wherein the step of scoring the patient data in reference to patient population data comprises the further step of scoring the data to yield a multi-dimensional indication of patient health.

14. The method of claim 12, wherein the step of scoring the patient data in reference to patient population data comprises the further step of scoring the data to predict a disease trend.

15. The method of claim 12, wherein the step of scoring the patient data in reference to patient population data comprises the further step of scoring the data to predict a next phase of disease progression.

16. The method of claim 12, wherein the step of scoring the patient data in reference to patient population data comprises the further step of scoring the data to predict co-morbidities.

17. The method of claim 12, wherein the step of scoring the patient data in reference to patient population data comprises the further step of scoring the data to infer other possible disease states.

18. The method of claim 12, wherein the step of scoring the patient data in reference to patient population data comprises the further step of scoring the data to predict a trend of patient health.

19. The method of claim 13, wherein the step of scoring the patient data in reference to patient population data comprises the further step of scoring the data to predict a disease trend.

20. The method of claim 13, wherein the step of scoring the patient data in reference to patient population data comprises the further step of scoring the data to predict a next phase of disease progression.

21. The method of claim 13, wherein the step of scoring the patient data in reference to patient population data comprises the further step of scoring the data to predict co-morbidities.

22. The method of claim 13, wherein the step of scoring the patient data in reference to patient population data comprises the further step of scoring the data to infer other possible disease states.

23. The method of claim 13, wherein the step of scoring the patient data in reference to patient population data comprises the further step of scoring the data to predict a trend of patient health.

24. The method of claim 1, wherein the step of analyzing the retrieved data comprises the further step of analyzing the retrieved data internal to the patient.

25. The method of claim 1, wherein the step of analyzing the retrieved data comprises the further step of analyzing the retrieved data external to the patient.

26. The method of claim 1, wherein the step of analyzing the retrieved data comprises the further step of analyzing the data to yield a multi-dimensional indication of patient health.

27. The method of claim 1, wherein the step of analyzing the retrieved data comprises the further step of analyzing the data using principles of fuzzy logic to yield a multi-dimensional indication of patient health.

28. The method of claim 26, wherein the step of analyzing the data comprises the further step of analyzing the data to predict a disease trend.

29. The method of claim 26, wherein the step of analyzing the data comprises the further step of analyzing the data to predict a next phase of disease progression.

30. The method of claim 26, wherein the step of analyzing the data comprises the further step of analyzing the data to predict co-morbidities.

31. The method of claim 26, wherein the step of analyzing the data comprises the further step of analyzing the data to infer other possible disease states.

32. The method of claim 26, wherein the step of analyzing the data comprises the further step of analyzing the data to predict a trend of patient health.

33. The method of claim 27, wherein the step of analyzing the data comprises the further step of analyzing the data to predict a disease trend.

34. The method of claim 27, wherein the step of analyzing the data comprises the further step of analyzing the data to predict a next phase of disease progression.

35. The method of claim 27, wherein the step of analyzing the data comprises the further step of analyzing the data to predict co-morbidities.

36. The method of claim 27, wherein the step of analyzing the data comprises the further step of analyzing the data to infer other possible disease states.

37. The method of claim 27, wherein the step of analyzing the data comprises the further step of analyzing the data to predict a trend of patient health.

38. The method of claim 1, wherein the step of communicating the sensed data, the analyzed data and the patient health evaluation comprises the further step of communicating the data and evaluation to the data management module for future analysis.

39. The method of claim 1, wherein the step of communicating the sensed data, the analyzed data and the patient health evaluation comprises the further step of communicating the data and evaluation to the data management module for access by a clinician.

40. The method of claim 1, wherein the step of communicating the sensed data, the analyzed data and the patient health evaluation comprises the further step of communicating a relative urgency of intervention.

41. The method of claim 40, wherein the step of communicating a relative urgency of intervention comprises the further step of communicating the relative urgency of intervention to a clinician.

42. The method of claim 40, wherein the step of communicating a relative urgency of intervention comprises the further step of communicating the relative urgency of intervention to a patient.

43. The method of claim 1, wherein the step of communicating the sensed data, the analyzed data and the patient health evaluation comprises the further step of communicating the data and evaluation to a data teaching system.

44. The method of claim 43, wherein the step of communicating the sensed data, the analyzed data and the patient health evaluation to the data teaching system comprises the further step of communicating the data and evaluation to a neural network.

45. The method of claim 44, wherein the step of communicating the sensed data, the analyzed data and the patient health evaluation to the neural network comprises the further step of verifying the data and evaluation for clinical accuracy and significance.

46. The method of claim 44, wherein the step of communicating the sensed data, the analyzed data and the patient health evaluation to the neural network comprises the further step of establishing a threshold of acceptable misidentifications.

47. The method of claim 44, wherein the step of communicating the sensed data, the analyzed data and the patient health evaluation to the neural network comprises the further step of establishing a threshold of acceptable misdiagnoses.

48. A method for using a data management system to automatically diagnose patient health comprising the steps of:
a. implanting a medical device in a patient;
b. populating a data management module adapted to store and archive data with patient population data to create a patient population database;
c. sensing data from the patient using the medical device;
d. delivering the sensed patient data from the medical device to the data management module to create a historical patient database;
e. retrieving the sensed patient data and the patient population data from the data management module for analysis;
f. analyzing the retrieved data using a neural network comprising clinically derived algorithms reflective of a standard of medical care to provide an initial evaluation of probable patient health based on the analyzed data; and
g. communicating the sensed data, the analyzed data and the patient health evaluation for access by a clinician;
h. accessing patient data from the neural network.

49. A data management system for automatic diagnosis of patient health comprising:
a. a sensing module adapted to sense data from a patient;
b. a data management module adapted to store and archive data;
c. an analysis module adapted to analyze data to make an initial evaluation of probable patient health by using clinically derived algorithms reflective of a standard of medical care; and
d. a communications module adapted to communicate the sensed data, the analyzed data and the patient health evaluation of patient health.

50. The data management system of claim 49, wherein the system comprises an electronic system.

51. The data management system of claim 49, wherein the system comprises a data teaching system.

52. The data teaching system of claim 50, wherein the system comprises an interactive neural network.

53. The neural network of claim 52, wherein the neural network is adapted to capture a time dependent dimension of disease states progression.

54. The neural network of claim 53, wherein the neural network can be partially trained with data.

55. The neural network of claim 53, wherein the neural network is untrained.

56. The neural network of claim 54, wherein the neural network is trained with data reflecting historical symptoms, diagnoses and outcomes, along with time development of the diseases and co-morbidities.

57. The neural network of claim 56, wherein the neural network is adapted to create new neural network coefficients that are distributable as a neural network knowledge upgrade.

58. The neural network of claim 52, wherein the neural network comprises databases of test cases, appropriate outcomes and the relative occurrence of misidentifications of the proper outcome or misdiagnoses.

59. The neural network of claim 52, wherein the neural network is adapted to establish a threshold of acceptable misidentifications or misdiagnoses.

60. The data management system of claim 49, wherein the system is configured as an Advanced Patient Management System.

61. The Advanced Patient Management System of claim 60, wherein the system comprises:
a. an implantable medical device further comprising the sensing module including sensors configured to monitor physiological functions;
b. the data management module configured to process data collected from the sensors and external input data;
c. the analysis module configured as an analytical engine adapted to combine device collected data with external input data to perform a predictive diagnosis; and
d. a therapeutic module configured to provide appropriate therapy based on the predictive diagnosis.

62. The data management system of claim 49, wherein the system comprises an implantable medical device.

63. The sensing module of claim 49, wherein the sensing module is internal to the patient.

64. The sensing module of claim 49, wherein the sensing module is external to the patient.

65. The data management module of claim 49, wherein the stored and archived data comprises the sensed patient data.

66. The data management module of claim 49, wherein the stored and archived data comprises patient population data.

67. The data management module of claim 66, wherein the patient population data comprises historical data from a patient.

68. The data management module of claim 66, wherein the patient population data comprises patient population data comprised of similarly sick patients.

69. The data management module of claim 66, wherein the patient population data comprises patient population data comprised of genetically similar patients.

70. The data management module of claim 49, wherein the data management module is adapted to store and archive data for future analysis.

71. The data management module of claim 49, wherein the data management module is adapted to store and archive data for access by the communications module.

72. The communications module of claim 49, wherein the communications module retrieves data from the data management module for analysis by the analysis module.

73. The communications module of claim 72, wherein the communications module retrieves data from the data management module periodically.

74. The analysis module of claim 49, wherein the analysis module is internal to the patient.

75. The analysis module of claim 49, wherein the analysis module comprises a neural network external to the patient.

76. The analysis module of claim 49, wherein the analysis module scores patient data retrieved from the data management module in reference to patient population data retrieved from the data management module.

77. The analysis module of claim 49, wherein the analysis module scores patient data retrieved from the data management module in reference to patient population data retrieved from the data management module using principles of fuzzy logic.

78. The analysis module of claim 76, wherein the scored patient data in reference to patient population data yields a multi-dimensional evaluation of patient health.

79. The analysis module of claim 77, wherein the scored patient data in reference to patient population data yields a multi-dimensional evaluation of patient health.

80. The analysis module of claim 78, wherein the scored patient data in reference to patient population data predicts a disease trend.

81. The analysis module of claim 78, wherein the scored patient data in reference to patient population data predicts a next phase of disease progression.

82. The analysis module of claim 78, wherein the scored patient data in reference to patient population data predicts co-morbidities.

83. The analysis module of claim 78, wherein the scored patient data in reference to patient population data infers other possible disease states.

84. The analysis module of claim 78, wherein the scored patient data in reference to patient population data predicts a trend of patient health.

85. The analysis module of claim 79, wherein the scored patient data in reference to patient population data predicts a disease trend.

86. The analysis module of claim 79, wherein the scored patient data in reference to patient population data predicts a next phase of disease progression.

87. The analysis module of claim 79, wherein the scored patient data in reference to patient population data predicts co-morbidities.

88. The analysis module Of claim 79, wherein the scored patient data in reference to patient population data infers other possible disease states.

89. The analysis module of claim 79, wherein the scored patient data in reference to patient population data predicts a trend of patient health.

90. The analysis module of claim 49, wherein the analysis module analyzes data retrieved from the data management module.

91. The analysis module of claim 49, wherein the analysis module analyzes data retrieved from the data management module using principles of fuzzy logic.

92. The analysis module of claim 90, wherein the analyzed data yields a multi-dimensional evaluation of patient health.

93. The analysis module of claim 91, wherein the analyzed data yields a multi-dimensional evaluation of patient health.

94. The analysis module of claim 92, wherein the analyzed data predicts a disease trend.

95. The analysis module of claim 92, wherein the analyzed data predicts a next phase of disease progression.

96. The analysis module of claim 92, wherein the analyzed data predicts co-morbidities.

97. The analysis module of claim 92, wherein the analyzed data infers other possible disease states.

98. The analysis module of claim 92, wherein the analyzed data predicts a trend of patient health.

99. The analysis module of claim 93, wherein the analyzed data predicts a disease trend.

100. The analysis module of claim 93, wherein the analyzed data predicts a next phase of disease progression.

101. The analysis module of claim 93, wherein the analyzed data predicts co-morbidities.

102. The analysis module of claim 93, wherein the analyzed data infers other possible disease states.

103. The analysis module of claim 93, wherein the analyzed data predicts a trend of patient health.

104. The communications module of claim 49, wherein the communications module is adapted to communicate the analyzed data to the data management module.

105. The communications module of claim 49, wherein the communications module is adapted to communicate the analyzed data for access by a clinician.

106. The communications module of claim 49, wherein the communications module is adapted to communicate a relative urgency of intervention based on the analyzed data.

107. The communications module of claim 105, wherein the relative urgency of intervention is communicated to a clinician.

108. The communications module of claim 105, wherein the relative urgency of intervention is communicated to a patient.

109. The communications module of claim 49, wherein the communications module is adapted to communicate the sensed data, the analyzed data and the patient health evaluation for access by a clinician.

110. The communications module of claim 49, wherein the communications module is adapted to communicate the sensed data, the analyzed data and the patient health evaluation to a data teaching system.

111. The data teaching system of claim 109, wherein the data teaching system comprises a neural network.

112. The communications module of claim 49, wherein the communications module is adapted to communicate the sensed data, the analyzed data and the patient health evaluation to a neural network to verify the data and evaluation for clinical accuracy and significance.

113. The communications module of claim 49, wherein the communications module is adapted to communicate the sensed data, the analyzed data and the patient health evaluation to a neural network to establish a threshold of acceptable misidentifications.

114. The communications module of claim 49, wherein the communications module is adapted to communicate the sensed data, the analyzed data and the patient health evaluation to a neural network to establish a threshold of acceptable misdiagnoses.

115. A data management system for automatic diagnosis of patient health comprising:
a. an implantable medical device further comprising;
i. a sensing module adapted to sense data from a patient;
ii. a data management module adapted to store and archive the patient data sensed by the sensing module to create a historical patient database and patient population data to create a patient population database;
iii. an analysis module adapted to analyze the historical patient data in comparision to the patient population data to make an initial evaluation of probable patient health by using clinically derived algorithms reflective of a standard of medical care;
iv. a communications module adapted to communicate the sensed data, the analyzed data and the patient health evaluation of patient health for access by a clinician; and
b. a neural network adapted to store patient data.
